(19) United States
(12) Patent Application Publication
(10) Pub. No.: US 2010/0197790 A1
(43) Pub. Date: Aug. 5, 2010

(54) CONTAINER FOR PHARMACEUTICAL USE FOR THE QUANTITATIVE RELEASE OF A SINGLE DOSE FOR ORAL ADMINISTRATION OF T3 AND T4 THYROID HORMONES IN SOLUTION

(75) Inventors: Giorgio Zoppetti, Milano (IT); Antonio Fontana, Carpi (IT)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Altergon S.A., Lugano (CH)

(21) Appl. No.: 12/388,691

(22) Filed: Feb. 19, 2009

(30) Foreign Application Priority Data
Jan. 30, 2009 (IT) MI2009A000118

(51) Int. Cl. A61K 31/197; A61K 47/32; A61K 47/40; A61P 5/14

(52) U.S. Cl. 514/567; 514/772.4; 514/777

(57) ABSTRACT

The present disclosure relates to a container for pharmaceutical use for the quantitative release of a single-dose for oral administration of the T3 and T4 thyroid hormones in solution, characterized by the fact of being formed with a plastic material having a Young's modulus between 10 and 80 MPa.

This application claims the benefit of Italian Patent Application No. M12009A000118, filed on 30 Jan. 2009, which is hereby incorporated by reference in its entirety.

The present disclosure relates to containers for pharmaceutical use, methods of administering pharmaceutical formulations with such containers, and pharmaceutical preparations using such containers.

The hormones produced by the thyroid cells are released into the blood stream and act on the body's metabolism by increasing oxygen consumption and heat production with an increase in body temperature, stimulate protein synthesis and make the nitrogen balance more positive, increase gluconeogenesis and glycogenolysis, and stimulate the synthesis, mobilization, and catabolism of cholesterol and lipids in general. The thyroid hormones increase the rate of oxidative cellular processes and regulate the metabolism of most tissues. In general, it has a predominantly anabolic effect at low doses, while it has a catabolic action at high doses. This biphasic action is evident in the metabolism of glycogen, proteins, and lipids.

In case of physiological deficiency, it is necessary to intervene with a therapy based on the administration of thyroid hormones. T3 (liothyronine, or O-(4-hydroxy-3-iodophenyl)-3,5-diiodo-L-tyrosine) and T4 (levothyroxine, or O-(4-hydroxy-3,5-diiodo phenyl)-3,5-diiodo-L-tyroxine are known thyroid hormones used, as such or in the form of sodium or hydrate salts, for various therapeutic applications and are obtained by synthesis or extraction from animal glands.

The therapeutic treatment of the deficiency of these hormones gives satisfactory results with the intake of T3 or T4 (or their respective salts, including, e.g., sodium or hydrate salts). In particular, T3 and T4 are used primarily in the treatment of hypothyroidism.

The thyroid hormone administration therapy can often last for the patient's entire life. The dosage must be individually determined. Generally, the initial dose is low. The amount is then gradually increased until the clinical evaluation and laboratory tests indicate that an optimal response has been obtained from the treated organism. The dose required to obtain this response is then maintained. The age and general physical condition of the patient and the severity and length of the hypothyroidism symptoms determine the initial dose and the speed with which the dosage can be brought to the definitive level. It is particularly important to only increase the doses very gradually in patients with myxedema or with cardiovascular diseases to prevent the manifestation of angina, myocardial infarction or stroke.

For these reasons, T3 and T4, their respective sodium salts, and their combination (Liotrix) are administered orally, in particular through tablets that allow their administration to be adapted to the patient's individual situation through the control of their ingestion frequency and through the choice of the dosage units.

A precise dosage is extremely critical as an underdosage could lead to an insufficient response and therefore to hypothyroidism. On the other hand, an overdosage would lead to toxic manifestations of hyperthyroidism including heart pain, palpitations, or cardiac arrhythmias. In patients with coronary disease, even a small increase in the dose of levothyroxine could be dangerous.

Therefore, due to risks associated with overdosage or underdosage of thyroid hormones in general, it is absolutely critical that patients can rely on formulations that are reliable in terms of titer and bioavailability.

Liothyronine (T3) and levothyroxine (T4) are currently on the market as oral drops in addition to a solid oral form primarily including tablets and soft gelatin capsules.

The former is a single container of 20 ml equipped with a dropper. However, this dropper does not guarantee the precise measurement of provided volume that is desired. Calculating a content of approximately 3.5 μg of T4 for every drop, various intermediate dosages between 3.5 and 200 μg could in theory be obtained. The latter dosage is reached by aliquoting 2 ml with 56 drops (considering the minimum quantity of a drop). However, dispensing a high number of drops is not easy or safe. Further, the drops are never dispensed at an identical volume in a repeatable way. Therefore, it is not generally possible to guarantee the dosing precision that is obtained with the individual formulation in tablets or soft capsules.

However, a significant advantage of the liquid formulation is the stability of the active ingredient for both T4 and for T3. Therefore, the ability to accurately deliver the volume of a solution containing the effective dose of liothyronine T3 and levothyroxine T4 to administer while maintaining the ideal stability of the active ingredient is the problem to be resolved by this disclosure.

The plastic containers available today for pharmaceutical use are primarily made of polyethylene and/or polypropylene, and are designed to dispense even viscous liquid by guaranteeing a minimum dispensable volume. However, they do not permit delivery of precise amounts. Even with single-dose bottles, the bottle does not allow a quantitative release of the contained dose, i.e. complete, but only the release of a minimum dose.

This may be acceptable in some therapeutic fields. For example, soft vials for ophthalmic use, and which contain eye washes or eye drops, generally do not need to provide a precise dosage of a drug or medicament. However, such imprecision is not acceptable for the case at hand, i.e. for thyroid hormones, for the reasons cited above. Bottles of the quantitative administration of thyroid hormones are not known, nor have they been proposed until now.

Therefore, one object of this disclosure is to provide a solution for the technical problem described above.

Accordingly, a container for pharmaceutical use is proposed for the quantitative release of a single-dose for oral administration of T3 and/or T4 thyroid hormones in solution, wherein the container comprises a plastic material having a Young's modulus between 10 and 80 MPa. In some non-limiting embodiments, the plastic material has a Young's modulus ranging from at least 10, 15, 20, 25, 30, 35, 40, 45, 50, 55, 60, 65, or 70 MPa, or more, to less than 80 MPa. In further non-limiting embodiments, the Young's modulus ranges, for example, from less than 80, 75, 70, 65, 60, 55, 50, 45, 40, 35, 30, or 25 MPa, or less, to greater than 10 MPa. Of course, the Young's modulus may be any range within the upper and lower bounds specified above, and be suitable for use in the present disclosure.

Therefore, according to the disclosure, suitable plastic materials are those which can be formed with a Young's modulus that is sufficiently low and within the range defined above. It has in fact been found that plastics having such a Young's modulus may be formed into containers, such as a bottle, and that such containers allow an almost complete emptying, or extraction, of a T3 and T4 thyroid hormone solution under compression. As will be demonstrated in Table 1 below, the range of Young's modulus suitable for the present disclosure is lower than the Young's modulus exhibited by conventional plastic materials (e.g., polyethylene and polypropylene) that are used to manufacture pharmaceutical containers, such as single-dose bottles for ophthalmic use containing eye washes or eye drops.

The Young's modulus of the samples described herein was determined using samples of the size specified by the UNI-EN-ISO 527-1 reference standard. The measured samples were subjected to traction with a traction velocity of 5 mm/min.

According to a non-limiting embodiment of the present disclosure, containers comprising plastic materials having a Young's modulus within the above-described range can be obtained by injection molding. In such a process, the Young's modulus of the suitable plastic material may range, for example, between 30 and 80 MPa.

As examples of plastic materials that are suitable for use in accordance with the present disclosure, non-limiting mention is made of polyethylene (“PE”), low-density polyethylene (“LDPE”), polypropylene (“PP”), ethylene-vinyl acetate (“EVA”), mixtures thereof, gelatin, and gelatin-containing materials. In a non-limiting embodiment of the present disclosure, the plastic material is a mixture of PE or PP with EVA, such as, for example, a mixture of 15% PR and 85% EVA, or a mixture of 35% PE and 65% EVA. In another non-limiting embodiment of the present disclosure, the plastic material is gelatin or a gelatin-containing material.

For the purposes of illustration, the Young's modulus was determined for two different materials according to the present disclosure, and is reported in Table 1 below. For comparative purposes, the Young's modulus for conventional PP and LDPE materials used in the formation of pharmaceutical containers is also reported.

TABLE 1
Young's Modulus/traction velocity of 5 mm/min (UNI-EN-ISO 527-1)

| SAMPLE | Young's Modulus (MPa) |
|---|---|
| PP* | 100.7 ± 3.3 |
| LDPE* | 92.9 ± 3.5 |
| 50% PE/50% EVA Mix | 63.5 ± 3.4 |
| 25% PE/75% EVA Mix | 42.2 ± 5.1 |

*comparative

According to a further embodiment of the present disclosure, plastic materials with a suitable Young's modulus include gelatin and mixtures thereof. The Young's modulus of such materials may, for example, range from between 10 and 50 MPa.

The plastic materials in accordance with the present disclosure may be manufactured into soft gelatin capsules. Such capsules may be produced, for example, by a rotary-die process. For example, soft capsules may be manufactured by injecting a T3 and/or T4 thyroid hormone solution into a gelatin-based plastic material that is in the form of a gelatinous semi-finished product in the fusion state. A sealable opening may be provided within the soft capsule for delivery of the solution.

The gelatin and gelatin-containing materials described herein may also be mixed with substances that make the gelatin insoluble in or impermeable to water. Non-limiting examples of such substances include cyclodextrins and dimethicone. Non-limiting mention is also made of polyvinyl alcohol (PVA), polyacrylates, and aluminum glycinate, which are useful substances for making gelatin insoluble in water.

Other processes known and described in the pharmaceutical literature for the production of soft elastic capsules (SEC) with liquid or semi-liquid content, such as the “Plate Process” or the use of the “Norton Capsule Machine” or the “Accogel Capsule Machine” as in “Remington's Pharmaceutical Sciences”, 18th edition, edited by Alfonso R. Gennaro, 1990, Mack Publishing Company, Easton Pa. 18042, ISBN 0-912734-04-3, are applicable for the production of containers in the form of soft capsules according to this disclosure including thyroid hormones and any excipients in a liquid or semi-liquid carrier.

The plastic materials for use in the present disclosure may also have a total free surface energy, according to the Owens Wendt method, within a particular range. For example, the total surface energy of the material may be less than 36, 35.5, 35, 34.5, 34, 33.5, 33, 32.5, 32, 31.5, 31, 30.5, or 30 mN/m, or less. Of course, the total surface energy of the plastic material may fall within any range specified by these upper and lower bounds. Thus, for example, the surface energy may range from 30-36 mN/m, 31-34 mN/m, 30-34 mN/m, or 30-32 mN/m. Containers formed from plastic materials having such surface energy may be effectively wetted by a T3 and T4 thyroid hormone solution. This allows such solution to flow within the container during the delivery in an ideal way, e.g., by allowing the flow of such solution along the inner wall of the container.

For illustrative purposes, the total surface energy values y (in mN/m), are provided in Table 2 below for two non-limiting examples of plastic materials suitable for use in the present disclosure.

| Material | Total Surface Energy (mN/m) |
|---|---|
| 50% PE/50% EVA Mix | 31.3 |
| 25% PE/75 EVA Mix | 34.1 |

Other than in the operating examples, or where otherwise indicated, all numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where a range is given, even if the term “between” is used, the ranges defined include the stated endpoints.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement. The examples that follow serve to illustrate the invention without, however, being limiting in nature.

The characteristics and advantages of this disclosure will now be demonstrated by the following non-limiting examples. Comparative example 2 is reported for purposes of comparison as an example using the known technique. In the following examples, the “emptying test” comprised weighing the liquid released by manually compressing the container.

For all patents, applications, or other reference cited herein, it should be understood that such documents are incorporated by reference in their entirety for all purposes, as well as for any specifically recited proposition. Where any conflict exists between a document incorporated by reference and the present application, this application will dominate.

### EXAMPLE 1

### Preparation of a Glycerol-Ethanol Solution of Levothyroxine Sodium (T4)

Components and quantities for a preparation of 25 liters were as follows:

Levothyroxine sodium (T4) 2.625 g

### Glycerol (85%) 21.525 kg

### Ethanol (96%) 6.100 kg

In a steel container of 10 liters equipped with a blade stirrer and cover, 90% of the ethanol (5.49 liters) was added and the T4 was added while stirring. The mixture was stirred slowly while maintaining a flow of nitrogen until complete dissolution was achieved. Glycerol (21.525 kg) was poured into a 25 liter turboemulsifier (Olsa-Italy). The ethanol solution containing the T4 solution was then added. The 10-liter container was washed with the remaining ethanol (0.61 liters) and poured it into the 25-liter turboemulsifier. The mixture was stirred at low speed for 15 minutes under nitrogen and protected from light.

### COMPARATIVE EXAMPLE 2

Preparation of neutral LDPE single-dose plastic containers, of 1.0 ml nominal (1.3 ml filling volume) with a screw cap containing the glycerol-ethanol solution of levothyroxine sodium (T4).

a) Preparation of Single-Use Containers

Material, quantity for preparation and relative percentage composition were as follows:

Low-density polyethylene (LDPE) 50.0 Kg 100%

A strip of 5 single-doses and the strip of 5 caps was produced by injection molding with two different molds, and then assembling with semi-automatic equipment. The product consisted of a strip of 5 1.0 ml single-doses with screw cap.

The Young's modulus of the molded products was carried out using samples of the size specified by the UNI-EN-ISO 527-1 reference standard. These samples were subjected to traction with a traction velocity of 5 mm/min. The measured Young's modulus is reported below

| Material | Young's Modulus |
|---|---|
| LDPE* | 92.9 MPa |

*Comparative

b) Preparation of the T4 Solution

The glycerol-ethanol solution obtained according to example 1 was used.

c) Filling of the Single-Use Containers

The containers obtained in a) were filled with 1.05 ml of the glycerol-ethanol solution described in b) by automatic pipette (Gilson P-1000), and then sealed with a Pentaseal-lab model bench-top sealer (Lameplast—Rovereto di Modena—Italy).

The emptying test was performed on a container according to c). The percentage extractability of the solution with respect to theory was 90%.

### EXAMPLE 3

Preparation of neutral LDPE/EVA single-dose plastic containers, of 1.0 ml nominal (1.3 ml filling volume) with screw cap containing the glycerol-ethanol solution of levothyroxine sodium (T4).

a. Preparation of the Single-Use Containers

Material, quantity for preparation and relative percentage composition were as follows:

Low-density polyethylene (LDPE) 25.0 kg 50%
ethylene-vinyl acetate (EVA) 25.0 kg 50%

A strip of 5 single-doses and the strip of 5 caps were produced by injection molding with two different molds, and then were assembled with semi-automatic equipment. The product consisted of a strip of 5 1.0 ml single-doses with screw cap.

The Young's modulus of the molded products was determined using samples of the size specified by the UNI-EN-ISO 527-1 reference standard. These samples were subjected to traction with a traction velocity of 5 mm/min. The measured Young's modulus is reported below

| Material | Young's Modulus |
|---|---|
| 50 LDPE/50 EVA Mix | 63.5 MPa |

The total surface energy y of the material was also evaluated according to the Owens Wendt method. The measure total surface energy is reported below:

| Material | Total Surface Energy |
|---|---|
| 50 LDPE/50 EVA Mix | 31.3 mN/m |

### b) Preparation of the T4 Solution

The glycerol-ethanol solution obtained according to example 1 was used.

### c) Filling of the Single-Use Containers

The containers obtained in a) were filled with 1.05 ml of glycerol-ethanol solution described in b) by automatic pipette (Gilson P-1000), and then sealed with a Pentaseal-lab model bench-top sealer (Lameplast—Rovereto di Modena—Italy).

The emptying test was performed on a container according to c). The percentage extractability of the solution with respect to theory was 96%.

### EXAMPLE 4

Preparation of neutral LDPE/EVA single-dose plastic containers, of 1.0 ml nominal (1.3 ml filling volume) with screw cap containing the glycerol-ethanol solution of levothyroxine sodium (T4).

### a) Preparation of the Single-Use Containers

Material, quantity for preparation and relative percentage composition were as follows:

Low-density polyethylene (LDPE) 12.5 kg 25%
ethylene-vinyl acetate (EVA) 37.5 kg 75%

A strip of 5 single-doses and the strip of 5 caps were produced by injection molding with two different molds, and then were assembled with semi-automatic equipment. The product consisted of a strip of 5 1.0 ml single-doses with screw cap.

The Young's modulus of the molded products was determined using samples of the size specified by the UNI-EN-ISO 527-1 reference standard were used. These samples were subjected to traction with a traction velocity of 5 mm/min. The measured Young's modulus is reported below:

| Material | Young's Modulus |
|---|---|
| 25 LDPE/75 EVA Mix | 42.2 MPa |

The total surface energy y of the material was also evaluated according to the Owens Wendt method. The measured total surface energy is reported below:

| Material | Total Surface Energy |
|---|---|
| 25 LDPE/75 EVA Mix | 34.1 mN/m MPa |

### b) Preparation of the T4 Solution

The glycerol-ethanol solution obtained according to example 1 was used.

### c) Filling of the Single-Use Containers

The containers obtained in a) were filled with 1.05 ml of glycerol-ethanol solution described in b) by automatic pipette (Gilson P-1000), and then sealed with a Pentaseal-lab model bench-top sealer (Lameplast—Rovereto di Modena—Italy).

The emptying test was performed on a container according to c). The percentage extractability of the solution with respect to theory was 98%.

### EXAMPLE 5

Preparation of Single-Dose Plastic Containers in the Form of Openable Soft gelatin capsules containing T4 in ethylene glycol and ethanol solution.

### a) Preparation of the Mixture for the Container's Casing

Components, quantity for preparation and relative percentage composition were as follows:

Gelatin 150 bloom 28.0 kg 35.0%
Sorbitol (special polyol solution) 5.6 kg 7.0%

### Dimethicone 1000 24.0 kg 30.0%

Purified water 22.4 kg 28.0%

In a 150-liter turboemulsifier (Olsa-Italy), 5.6 kg of special sorbitol and 24 kg of dimethicone were added to 22.4 kg of purified water. Vigorous stirring was maintained and the temperature was brought to 70° C., and then 28 kg of gelatin were added and maintained under stirring for 15-60 minutes. The mass was then deaerated by applying a progressive vacuum until reaching a value between −0.8 and −0.9 bar. The mixture obtained was unloaded and stored, until the encapsulation, at the appropriate temperature, between 50° C. and 70° C.

### b) Preparation of the T4 Solution

The glycerol-ethanol solution obtained according to example 1 was used.

c) Preparation of the Container with Solution in the Form of Soft Capsules

Soft gelatin capsules with an 8-tube format (or twist-off) were prepared according to a known Rotary Die type process. Specifically, a gelatinous mixture prepared according to a) was transferred by nitrogen pressure to two thermostated (50° C./70° C.) spreader boxes. The mixture was then dripped onto two rollers cooled to 18° C.±5° C., resulting in the formation of gelatin ribbons of a predetermined thickness.

The two gelatin ribbons were accompanied to the sides of the solution injection segment and through two molds. In this phase, an injection pump injected a solution according to b), allowing the formation of the capsules.

The solution according to b) was injected in the measure of 1 ml, in capsules of an 8-tube format. The tubes were sealed by the combined and simultaneous pressure of the molds, the heating of the injection segment, and the ribbons (partial fusion).

The capsules formed were transferred to tumble driers where they began the water loss phase, which was completed after a pause in a desiccation tunnel. The moisture content of the capsules ranged from between 5% and 15%.

Openable soft gelatin capsules having the following characteristics were thus obtained:

average weight per capsule: 745 mg±7.5%
residual moisture: 1.0%
T4 content: 0.050 mg/capsule, equal to 100.0% d.d.
hardness: 6-10 N
Young's modulus: between 10 and 50 MPa.

The emptying test was performed on a soft capsule container according to this example. The percentage extractability of the solution with respect to theory was 98%.

As can be understood from the entire description reported above, the present disclosure allows the achievement of a nearly quantitative release of a predetermined dose of thyroid hormones T3 and T4 in solution for oral administration, thus effectively achieving the originally proposed purpose.

1. A container for pharmaceutical use for the quantitative release of a single-dose for oral administration of T3 and/or T4 thyroid hormone(s) in solution, wherein the container comprises a plastic material having a Young's modulus between 10 and 80 MPa, said container being capable of providing a substantially quantitative release of said single-dose when subject to manual compression.

2. The container according to claim 1, wherein the plastic material has a Young's modulus between 30 and 80 MPa.

3. The container according to claim 2, wherein the plastic material is a mixture of polyethylene (PE) or polypropylene (PP) with ethylene-vinyl acetate (EVA).

4. The container according to claim 3, wherein said PE or PP is present in said mixture in an amount ranging from 15% to 35%, and said EVA is present in said mixture in an amount ranging from 85% to 65%.

5. The container according to claim 4, wherein said mixture is chosen from a mixture of 50% PE and %0% EV having a Young's Modulus of 63.5+/−3.4 MPa and a mixture of 25% PE and 755 EVA having a young's modulus of 42.2+/−5.1 MPa.

6. The container according to claim 1, wherein the plastic material comprises gelatin or a mixture comprising gelatin, said plastic material having a Young's modulus between 10 and 50 MPa.

7. The container according to claim 6, wherein the plastic material is a mixture of gelatin, water, and at least one additional substance, said at least one additional substance being chosen from agents that make the gelatin insoluble or impermeable to water.

8. The container according to claim 7, wherein said agents are chosen from cyclodextrins, dimethicone, polyvinyl alcohol (PVA), polyacrylates, and aluminum glycinate.

9. The container according to claim 7, wherein the plastic material is a mixture of gelatin, Sorbitol, Dimethicone, and water.

10. The container according to claim 1, wherein the plastic material has a surface energy under 36 mN/m.

11. A method for the preparation of a single-dose for oral administration of T3 and/or T4 thyroid hormone(s) in solution within a container according to claim 6, wherein the solution is injected within the gelatin-based plastic material in a gelatinous semi-finished form in a fusion state to give a soft capsule container according to the rotary die process; and wherein a sealable opening for the delivery of said solution for administration is provided in said soft capsule container.

12. A solution of T3 and T4 thyroid hormones for oral administration prepared in a soft capsule container according to the method of claim 11, said soft capsule being suitable for a single-use application.

13. A single-dose for the oral administration of a solution of T3 and T4 thyroid hormones, said solution being contained in a container comprising a plastic material having a Young's modulus between 10 and 80 MPa, wherein said container is capable of providing a substantially quantitative release of said hormones in solution when subject to manual compression.

14. The single-dose of claim 13, wherein the plastic material has a surface energy of less than 36 mN/m.

15. A method of administering a single-dose of a solution of T3 and/or T4 thyroid hormone to a patient in need thereof, comprising:
providing a container comprising a plastic material having a Young's modulus between 10 and 80 MPa, said container including said single-dose; and
orally administering a substantially quantitative amount of said single-dose from said container to said patient by compressing said container.

16. The method of claim 17, wherein the patient is a mammal.

17. The method of claim 18, wherein the mammal is a human.

18. The method of claim 17, wherein said substantially quantitative amount of said single dose provides a pharmaceutically effective amount of said solution of T3 and/or T4 thyroid hormone.

19. The method of claim 15, wherein said patient has hypothyroidism, or is at risk for a disease or disorder associated with hypothyroidism.

20. The method of claim 15, wherein compressing said container comprises manual compression.
